(19) United States
(12) Patent Application Publication
(10) Pub. No.: US 2007/0178126 A1
(43) Pub. Date: Aug. 2, 2007

(54) Polyamino acids functionalized by at least one (oligo)amino acid group and therapeutic uses

(75) Inventors: Stephanie Angot, Lyon (FR); You-Ping Chan, Lyon (FR); Gerard Soula, Meyzieu (FR)

Correspondence Address: PATTON BOGGS LLP, 8484 WESTPARK DRIVE, SUITE 900, MCLEAN, VA, 22102, US

(73) Assignee: Flamel Technologies, Venissieux (FR)

(21) Appl. No.: 11/601,691

(22) Filed: Nov. 20, 2006

Related U.S. Application Data
(63) Continuation of application No. 10/537,550, now abandoned.

(30) Foreign Application Priority Data
Dec. 4, 2002 (FR) 02/015269

(51) Int. Cl. A61K 8/02

(52) U.S. Cl. 424/401

(57) ABSTRACT

The invention relates to novel biodegradable polyamino acid based materials which can be used for the vectorization of (an) active substance(s) (PA). The invention also relates to novel pharmaceutical, cosmetic, dietary or phytosanitary compositions based on said polyamino acids. The present invention which primarily relates to polyamino acids comprising aspartic units and/or glutamic unites some of which bearing at least one graft, characterized in that at least one of said grafts is joined to an aspartic or glutamic unite by means of an amide bond and in that at least one of said grafts comprises at least one oligoamino acid which is Leu, and/or Ileu, and/or Val, and/or Phe based. Said amide function ensure better stability with respect to hydrolysis than similar products of prior art. Advantageously, said polymers can be easily and economically transformed into active substance vectorization particles, said particles being able to form stable aqueous colloidal suspensions.

The present invention relates to novel materials based on biodegradable polyamino acids that are useful especially for the vectorization of active principle(s) (AP).

The invention further relates to novel pharmaceutical, cosmetic, dietetic or phytosanitary compositions based on these polyamino acids. These compositions can be of the types that allow the vectorization of AP and preferably take the form of emulsions, micelles, particles, gels, implants or films.

The AP considered are advantageously biologically active compounds capable of being administered to an animal or human organism by the oral, parenteral, nasal, vaginal, ocular, subcutaneous, intravenous, intramuscular, intradermal, intraperitoneal, intracerebral or buccal route, etc.

The AP to which the invention relates more particularly, but without implying a limitation, are proteins, glycoproteins, peptides, polysaccharides, lipopolysaccharides, oligonucleotides or polynucleotides, and organic molecules. However, the invention can also relate to cosmetic products or to phytosanitary products such as herbicides, insecticides, fungicides, etc.

In the field of the vectorization of active principles, especially medicinal active principles, there is a need in many cases to:
protect them from degradation (hydrolysis, precipitation at the site, enzymatic digestion, etc.) until they reach their site of action,
and/or control their release rate so as to maintain a therapeutic level over a defined period,
and/or convey them (with protection) to the site of action.

Several types of polymers have been studied for these purposes and some are even available commercially. Examples which may be mentioned are polymers of the polylactic, polylactic-glycolic, polyoxyethylene-oxypropylene, polyamino acid or polysaccharide type. These polymers constitute starting materials for the manufacture e.g. of mass implants, microparticles, nanoparticles, vesicles, micelles or gels. Apart from the fact that these polymers have to be suitable for the manufacture of such systems, they also have to be biocompatible, non-toxic, non-immunogenic and economic and they must be easily removable from the body and/or biodegradable. On this last point, it is additionally essential that biodegradation in the organism generates non-toxic products.

Various patents, patent applications or scientific articles are referred to below in order to illustrate the prior art concerning polymers employed as starting materials for the production of AP vectorization systems.

U.S. Pat. No. 4,652,441 describes polylactide microcapsules encapsulating the hormone LH-RH. These microcapsules are produced by preparing a water-in-oil-in-water emulsion and comprise an aqueous inner layer containing the hormone, a substance (gelatin) for fixing the latter, an oily layer of polylactide and an aqueous outer layer (polyvinyl alcohol). The AP can be released over a period of more than two weeks after subcutaneous injection.

U.S. Pat. No. 6,153,193 describes compositions based on amphiphilic polyoxyethylene-polyoxypropylene micelles for the vectorization of anticancer agents such as adriamycin.

Akiyoshi et al. (J. Controlled Release 1998, 54, 313-320) describe pullulans which are rendered hydrophobic by the grafting of cholesterol and which form nanoparticles in water. These nanoparticles, which are capable of complexing reversibly with insulin, form stable colloidal suspensions.

U.S. Pat. No. 4,351,337 describes amphiphilic copolyamino acids based on leucine and glutamate which can be used in the form of implants or microparticles for the controlled release of active principles. The latter can be released over a very long period that depends on the degradation rate of the polymer.

U.S. Pat. No. 4,888,398 describes polymers based on polyglutamate or polyaspartate, and optionally polyleucine, with pendent groups of the alkoxy-carbonylmethyl type located randomly along the polyamino acid chain. These polyamino acids, grafted with side groups, e.g. methoxycarbonylmethyl groups, can be used in the form of biodegradable implants containing a sustained-release AP.

U.S. Pat. No. 5,904,936 describes nanoparticles obtained from a polyleucine-polyglutamate block polymer which are capable of forming stable colloidal suspensions and of associating spontaneously with biologically active proteins without denaturing them. The latter can then be released in vivo in a controlled manner over a long period.

U.S. Pat. No. 5,449,513 describes amphiphilic block copolymers comprising a polyoxyethylene block and a polyamino acid block, for example poly(beta-benzyl-L-aspartate). These polyoxyethylene-polybenzylaspartate polymers form micelles that are capable of encapsulating hydrophobic active molecules such as adriamycin or indomethacin.

Patent application WO-A-99/61512 describes polylysines and polyornithines functionalized by a hydrophobic group (palmitic acid bonded to polylysine or ornithine) and a hydrophilic group (polyoxyethylene). In the presence of cholesterol, these polymers, for example polylysine grafted with polyoxyethylene and palmitoyl chains, form vesicles capable of encapsulating doxorubicin or DNA. These polymers based on polylysines are cationic in a physiological medium.

Patent application WO-A-02/28251, in the name of the Applicant, relates to a suspension of biocompatible vectorization particles (VP) for active principles (AP). These VP are based on a hydrophilic neutral polyamino acid poly(AANI)/hydrophobic neutral polyamino acid poly(AANO) diblock copolymer. In colloidal suspension in the undissolved state, these particles of poly(AANI)/poly(AANO) are capable of associating at least one AP and releasing it, especially in vivo, in a sustained and/or delayed manner. These novel VP form stable aqueous suspensions spontaneously and without the aid of surfactants or organic solvents. The hydrophilic neutral polyamino acid poly(AANI)/hydrophobic neutral polyamino acid poly(AANO) diblock copolymer can be e.g. poly(Gln-N-hydroxyethyl)/poly(Leu) derived from the aminolysis of poly(Glu-O-alkyl)/poly(Leu) with hydroxyethylamine.

These copolymers are neutral in a physiological medium.

Patent application WO-A-00/30618, in the name of the Applicant, describes poly(sodium glutamate)/poly(methyl, ethyl, hexadecyl or dodecyl glutamate) block or random polymers capable of forming stable colloidal suspensions and of associating spontaneously with biologically active proteins without denaturing them. The latter can then be released in vivo in a controlled manner over a long period. These amphiphilic copolyamino acids are modified by the presence of a hydrophobic alkyl side chain. These alkyl groups are covalently grafted onto the polymer via an ester group. These polymers are anionic in a physiological medium.

They are capable of improvement in at least two respects, depending on the intended application:
[symbol] the relative stability of the ester group in an aqueous medium,
[symbol] and the use of certain non-natural alcohols, such as hexanol, as precursors of hydrophobic alkyl grafts. The latter aspect is particularly problematic in terms of toxicity if the concentration of polymer laden with these residual alcohols becomes large.

As regards the state of the art relating to branched polyamino acids which are described in the literature and functionalized by oligoamino acids, the following works are of note:

Patent WO-A-87/03891 describes polyglutamates or polyaspartates carrying diacid groups of the malonic or succinic type that are bonded to the polyamino acid chain via a rotating linkage (“spacer”) of oligopeptide character. The presence of the diacid group makes it possible to fix calcium cations or form cyclic anhydrides capable of reacting with an active principle. These polymers can be used particularly in the form of implants for the slow release of an active principle in vivo. In the same spirit, Hoes et al. [J. Controlled Release 1 (1985) 301-315 & 2 (1985) 205-213] describe polyglutamates in which an anticancer compound (adriamycin) is grafted onto the polymer via a glycine-glycine-leucine rotating linkage (“spacer”) that is readily degraded in vivo.

In another context, branched polyamino acids based on polylysine have been synthesized for their evaluation in immunology (Hudecz et al., Polymeric Materials in Medication, Plenum Press, New York, 1985, pages 265-289) or for physical studies (Mezo et al., J. Controlled Release 2000, 63, 81-95). These polymers have a polylysine skeleton and each lysine unit is connected to a hydrophilic oligopeptide.

Said document does not teach the use of these polymers for associating and/or vectorizing active principles not bonded to the polymers.

Thus, even though a very large number of technical solutions exist in the prior art that have been developed and proposed for the vectorization of medicinal active principles, the answer to the demands as a whole is difficult to achieve and remains unsatisfactory. More specifically, the idea of a polyamino acid grafted with (oligo)amino acids that is capable of forming a stable colloidal aqueous suspension of vectorization particles able to associate reversibly with active principles has not been described hitherto.

In this context, one of the essential objects of the present invention is to provide a novel family of polymers that are anionic at animal physiological pH (e.g. in the order of 7.4) and based on polyglutamate and polyaspartate, said polymers representing an improvement compared with the polymers described in patent application WO-A-00/30618, especially in terms of stability and non-toxicity.

According to another essential object of the present invention, these polymers should be capable of being used for the vectorization of AP and should make it possible optimally to satisfy all the specifications of the specifications sheet and the following in particular:
capacity to:
   easily and economically form stable aqueous colloidal suspensions,
   easily associate with numerous active principles,
   and release these active principles in vivo,
biocompatibility,
biodegradability,
stability to hydrolysis.

This and other objects are achieved by the present invention, which relates first and foremost to a polyamino acid comprising aspartic units and/or glutamic units, some of which carry at least one graft, characterized in that:
at least one of these grafts is bonded to an aspartic or glutamic unit via an amide linkage,
at least some of these grafts comprise one or more (oligo)amino acids, excluding the grafts carrying at least one carboxylic diacid cyclizable to an anhydride,
and the “amino acid” unit(s) in the (oligo)amino acid is (are) selected from those having an alkyl or aryl group in the alpha position, and preferably from those belonging to the group comprising alanine, valine, leucine, isoleucine and phenylalanine.

It is to the Applicant's credit to have had the idea of combining, in a totally judicious and advantageous manner, particular biodegradable and anionic polyAsp and/or polyGlu polyamino acids with grafts that contain at least one (oligo)amino acid unit and are bonded to the polyAsp and/or polyGlu skeleton via an amide linkage.

These novel (co)polymers have proved particularly suitable for the vectorization of proteins.

In one preferred embodiment of the invention, each graft is bonded to an aspartic or glutamic unit via an amide linkage.

In terms of the invention, the words “polyamino acid” cover both oligoamino acids comprising from 2 to 20 “amino acid” units and polyamino acids comprising more than 20 “amino acid” units.

Preferably, the oligoamino acid or (oligo)amino acids in all or some of the grafts consists (each consist) of mutually identical “amino acid” units.

According to one preferred characteristic of the invention, the number of “amino acid” units per graft varies from 1 to 6.

It is self-evident that, according to the invention, the constituent “amino acid” units of the grafts can be identical to or different from one another.

Compared with analogous products, these polymers have surprising properties of association and/or encapsulation with one or more active principles. Furthermore, they are easily degraded, in the presence of enzymes, to non-toxic catabolites/metabolites (amino acids).

In terms of the invention and throughout the present disclosure, the words “association” and “associate” employed to qualify the relationships between one or more active principles and the polyamino acids mean in particular that the active principle(s) is (are) bonded to the polyamino acid(s) especially by a weak bond, for example an ionic bond, and/or by hydrophobic contact, and/or are encapsulated by the polyamino acid(s).

Preferably, the polyamino acids according to the present invention are oligomers or homopolymers comprising glutamic or aspartic acid repeat units or copolymers comprising a mixture of these two types of “amino acid” units. The units in question in these polymers are amino acids having the D, L or D/L configuration and are bonded via their alpha or gamma positions in the case of the glutamate or glutamic unit and via their alpha or beta positions in the case of the aspartic or aspartate unit.

The preferred “amino acid” units in the main polyamino acid chain are those having the L configuration and a linkage of the alpha type.

Even more preferably, the polyamino acids according to the invention have general formula (I) below: 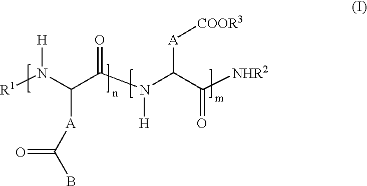
in which:
R¹ is H, a linear C2 to C10 alkyl or branched C3 to C10 alkyl, a benzyl or a terminal “amino acid” unit;
R² is H, a linear C2 to C10 acyl or branched C3 to C10 acyl group or a pyroglutamate;
R³ is H or a cationic entity preferably selected from the group comprising:
metal cations advantageously selected from the subgroup comprising sodium, potassium, calcium and magnesium,
organic cations advantageously selected from the subgroup comprising:
   cations based on amine,
   cations based on oligoamine,
   cations based on polyamine (polyethylenimine being particularly preferred),
   and cations based on amino acid(s) advantageously selected from the class comprising cations based on lysine or arginine,
and cationic polyamino acids advantageously selected from the subgroup comprising polylysine and oligolysine;
the n groups B independently of one another are each a monovalent radical of the following formula:

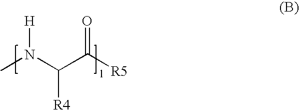

in which:
R⁴ is a methyl (alanine), isopropyl (valine), isobutyl (leucine), sec-butyl (isoleucine) or benzyl (phenylalanine), the amino acids given in brackets being those which correspond to the “amino acid” unit formed when R⁴ is the alkyl in question;
and R⁵ is a group selected from OH, NH₂, a C1 to C5 alkoxy group and a benzyloxy;
A independently is —CH₂— (aspartic unit) or —CH₂—CH₂— (glutamic unit);
n/(n+m) is defined as the molar grafting rate and varies from 0.5 to 100 mol %;
n+m varies from 3 to 1000 and preferably between 30 and 300;
and l varies from 1 to 6.

Advantageously, the length of the graft chain (β), which is determined on the one hand by the value of l and on the other hand by the choice of alkyl unit R⁴, make it possible to regulate the hydrophilic/lipophilic balance of the polymer according to the intended application.

In a first embodiment of the invention, the polyamino acids are alpha-L-glutamate or alpha-L-glutamic homopolymers.

In a second embodiment of the invention, the polyamino acids are alpha-L-aspartate or alpha-L-aspartic homopolymers.

In a third embodiment of the invention, the polyamino acids are alpha-L-aspartate/alpha-L-glutamate or alpha-L-aspartic/alpha-L-glutamic copolymers.

Advantageously, the distribution of the aspartic and/or glutamic units in the main polyamino acid chain is such that the resulting polymers are either random or of the block type or of the multiblock type.

Defined in another way, the polyamino acids according to the invention have a molecular weight of between 2000 and 100,000 g/mol and preferably of between 5000 and 40,000 g/mol.

As a further preference, the molar grafting rate of (oligo)amino acid units in the polyamino acids according to the invention should be between 2 and 70% and preferably between 5 and 40%.

Remarkably, the polyamino acids of the invention can be used in several ways according to the grafting rate. The methods of shaping a polymer for the encapsulation of an active principle in the various forms to which the invention relates are known to those skilled in the art. Further details can be obtained e.g. by consulting the few particularly pertinent references given below:
“Microspheres, Microcapsules and Liposomes; vol. 1. Preparation and chemical applications”, Ed. R. Arshady, Citus Books 1999. ISBN: 0-9532187-1-6.
“Sustained-Release Injectable Products”, Ed. J. Senior and M. Radomsky, Interpharm Press 2000. ISBN: 1-57491-101-5.
“Colloidal Drug Delivery Systems”, Ed. J. Kreuter, Marcel Dekker, Inc. 1994. ISBN: 0-8247-9214-9.
“Handbook of Pharmaceutical Controlled Release Technology”, Ed. D. L. Wise, Marcel Dekker, Inc. 2000. ISBN: 0-8247-0369-3.

Polyamino acids are also extremely valuable in that, with a relatively low grafting rate in the order of 3 to 30% (variable depending on the chosen (oligo)amino acid), they disperse in water at pH 7.4 (e.g. with a phosphate buffer) to give colloidal solutions or suspensions, or gels, according to the polymer concentration and the grafting rate. Furthermore, polyamino acids (in particulate or non-particulate form) can encapsulate or associate easily with active principles such as proteins, peptides or small molecules. The preferred shaping operation is that described in patent application WO-A-00/30618 in the name of the Applicant, which consists in dispersing the polymer in water and incubating the solution in the presence of an AP. This colloidal solution of vectorization particles consisting of the polyamino acids according to the invention can subsequently be filtered on a 0.2 μm filter and then injected directly into a patient.

Beyond a grafting rate of 30%, depending on the chosen (oligo)peptide, this particulate form according to patent application WO-A-00/30618 can be envisaged in particular in the case in point. The polymer can then form microparticles capable of associating or encapsulating AP. In this context the microparticles can be shaped by cosolubilizing the AP and the polymer in an appropriate organic solvent and then precipitating the mixture in water. The particles are subsequently recovered by filtration and can then be used for administration by the oral route (in the form of gelatin capsules, in a compacted and/or coated form, or else in a form dispersed in an oil) or by the parenteral route, after redispersion in water.

At grafting rates in excess of 50%, redispersion of the polymer in an aqueous phase becomes more difficult because of the smaller amount of ionizable carboxylate groups, and the polymer precipitates. In this case the polymer can be solubilized in a biocompatible solvent, such as N-methylpyrrolidone, or an appropriate oil, such as Mygliol®, and then injected by the intramuscular or subcutaneous route or into a tumor. Diffusion of the solvent or oil leads to precipitation of the polymer at the injection site and thus forms a deposit. These deposits then assure a controlled release of the polymer by diffusion and/or by erosion and/or by hydrolytic or enzymatic degradation.

Independently of the fact that the microparticulate form of the polyamino acid according to the invention is preferred, the polymers of the invention, in neutral or ionized form, can more generally be used by themselves or in a liquid, solid or gel composition and in an aqueous or organic medium.

It should be understood that the polymer based on polyamino acids contains carboxyl groups which are either neutral (COOH form) or ionized (COO⁻ anion), depending on the pH and the composition. For this reason the solubility in an aqueous phase is a direct function of the proportion of free COOH in the polymer (not grafted with the hydrophobic unit) and of the pH. In aqueous solution the countercation can be a metal cation such as sodium, calcium or magnesium, or an organic cation such as triethanolamine, tris(hydroxymethyl)aminomethane or a polyamine like polyethylenimine.

The polymers of the invention are obtained e.g. by methods known to those skilled in the art. The polyamino acids can be obtained by grafting the (oligo)amino acid directly onto the polymer by means of a conventional coupling reaction.

For example, a homopolyglutamate or homopolyaspartate polyamino acid or a block, multiblock or random glutamate/aspartate copolymer is prepared by conventional methods.

To obtain a polyamino acid of the alpha type, the most common technique is based on the polymerization of amino acid N-carboxy anhydrides (NCA), which is described e.g. in the article “Biopolymers” 1976, 15, 1869, and in the work by H. R. Kricheldorf entitled “Alpha-amino acid N-carboxy anhydride and related heterocycles”, Springer Verlag (1987). The NCA derivatives are preferably NCA-O-Me, NCA-O-Et or NCA-O-Bz derivatives (Me=methyl, Et=ethyl and Bz=benzyl). The polymers are then hydrolyzed under appropriate conditions to give the polymer in its acid form. These methods are based on the description given in patent FR-A-2 801 226 in the name of the Applicant. A number of polymers that can be used according to the invention, for example of the poly(alpha-L-aspartic), poly(alpha-L-glutamic), poly(alpha-D-glutamic) and poly(gamma-L-glutamic) types of variable molecular weights, are commercially available. The polyaspartic polymer of the alpha-beta type is obtained by the condensation of aspartic acid (to give a polysuccinimide) followed by basic hydrolysis (cf. Tomida et al., Polymer 1997, 38, 4733-36).

Coupling of the (oligo)amine with an acid group of the polymer is easily effected by reacting the polyamino acid in the presence of a carbodiimide as coupling agent, and optionally a catalyst such as 4-dimethylaminopyridine, in an appropriate solvent such as dimethylformamide (DMF), N-methylpyrrolidone (NMP) or dimethyl sulfoxide (DMSO). The carbodiimide is e.g. dicyclohexyl-carbodiimide or diisopropylcarbodiimide. The grafting rate is controlled chemically by the stoichiometry of the constituents and reactants or by the reaction time. The (oligo)amino acids can be obtained by sequential synthesis according to conventional methods (cf., for example, the: work entitled “Principles of Peptide Synthesis” by Bodanszky, Springer-Verlag 1984) or are commercially available.

According to another of its features, the invention relates to a pharmaceutical, cosmetic, dietetic or phytosanitary composition comprising at least one polyamino acid as defined above and optionally at least one active principle, which can be a therapeutic, cosmetic, dietetic or phytosanitary active principle.

According to yet another of its features, the invention relates especially to a pharmaceutical, cosmetic, dietetic or phytosanitary composition comprising at least one polyamino acid containing aspartic units and/or glutamic units, some of which carry at least one graft:
at least one of these grafts being bonded to an aspartic or glutamic unit via an amide linkage,
at least some of these grafts comprising one or more (oligo)amino acids,
and the grafts carrying at least one carboxylic diacid cyclizable to an anhydride being excluded,
   characterized in that it comprises at least one active principle associated with the polyamino acid(s) by one or more bonds other than one or more covalent chemical bonds.

Preferably, the active principle is a protein, a glycoprotein, a protein bonded to one or more polyalkylene glycol chains (preferably polyethylene glycol (PEG) chains: “PEGylated protein”), a polysaccharide, a liposaccharide, an oligonucleotide, a polynucleotide or a peptide.

Even more preferably, the active principle is a small hydrophobic, hydrophilic or amphiphilic organic molecule.

This composition can be in the form of nanoparticles, microparticles, emulsions, gels, micelles, implants, powders or films.

In one of its particularly preferred forms, the composition, whether or not laden with active principle(s), is a stable colloidal suspension of nanoparticles and/or microparticles and/or micelles of polyamino acids in an aqueous phase.

If the composition according to the invention is a pharmaceutical composition, it can be administered by the oral, parenteral, nasal, vaginal, ocular, subcutaneous, intravenous, intramuscular, intradermal, intraperitoneal, intracerebral or buccal route.

It is also possible to envisage a composition in the form of a solution in a biocompatible solvent that can be injected by the subcutaneous or intramuscular route or into a tumor.

In another variant, the composition according to the invention is formulated in such a way that it is capable of forming a deposit at the injection site.

The invention further relates to compositions which comprise polyamino acids according to the invention and AP and which can be used for the preparation of:
drugs, particularly for administration by the oral, nasal, vaginal, ocular, subcutaneous, intravenous, intramuscular, intradermal, intraperitoneal or intracerebral route, it being possible in particular for the active principles of these drugs to be proteins, glycoproteins, proteins bonded to one or more polyalkylene glycol chains {e.g. polyethylene glycol (PEG) chains, in which case the term “PEGylated” proteins is used}, peptides, polysaccharides, liposaccharides, oligonucleotides, polynucleotides and small hydrophobic, hydrophilic or amphiphilic organic molecules;
and/or nutriments;
and/or cosmetic or phytosanitary products.

This preparation is characterized in that it consists essentially in using at least one of the polyamino acids according to the invention, as defined above, and/or the compositions also described above.

The techniques of associating one or more AP with the grafted polyamino acids according to the invention are described especially in patent application WO-A-00/30618. They consist in incorporating at least one active principle into the liquid medium containing VP to give a colloidal suspension of VP laden or associated with one or more active principles, AP. This incorporation, which results in the AP being trapped by the VP, can be effected as follows:
by introduction of the AP into aqueous solution and then addition of the VP, either in the form of a colloidal suspension or in the form of isolated VP (lyophilizate or precipitate);
or by addition of the AP, either in solution or in the pure or preformulated state, to a colloidal suspension of the VP, optionally prepared immediately before use by dispersing the dry VP in an appropriate solvent such as water.

The invention further relates to a method of therapeutic treatment that consists essentially in administering the composition as described in the present disclosure by the oral, parenteral, nasal, vaginal, ocular, subcutaneous, intravenous, intramuscular, intradermal, intraperitoneal, intracerebral or buccal route.

The invention further relates to a method of therapeutic treatment that consists essentially in using a composition as described above, in the form of a solution in a biocompatible solvent, and then injecting it by the subcutaneous or intramuscular route or into a tumor, preferably in such a way that it forms a deposit at the injection site.

The following may be mentioned as examples of AP that can be associated with the polyamino acids according to the invention, whether or not they are in the form of nanoparticles or microparticles:
proteins such as insulin, interferons, growth hormones, interleukins, erythropoietin or cytokines;
peptides such as leuprolide or cyclosporin;
small molecules such as those belonging to the anthracycline, taxoid or camptothecin family;
and mixtures thereof.

The invention will be better understood and its advantages and variants will become clearly apparent from the Examples below, which describe the synthesis of the polyamino acids grafted with an (oligo)amino acid group, their conversion to an AP vectorization system (stable aqueous colloidal suspension) and the demonstration of the ability of such a system to associate with AP (small organic molecules, proteins, etc.) to form pharmaceutical compositions.

### EXAMPLE 1

### Preparation of Polymer P1

### Synthesis of a Polyglutamate Grafted with a Trileucine amide graft

1/ Structure of the Graft (Leu)₃NH₂:—RNCAS 73237-77-1 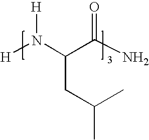
marketed by SIGMA
2/ Synthesis of the Polymer:
4 g of an alpha-L-polyglutamate (having a molecular weight equivalent to about 12,000 g/mol, relative to a polyoxyethylene standard, and obtained by the polymerization of monomers consisting of N-carboxy anhydride derivatives of methyl glutamate: NCAGluOMe, followed by hydrolysis, as described in patent application FR-A-2 801 226) are solubilized in 77 ml of dimethylformamide (DMF) by heating at 80° C. for 2 hours. Once the polymer is solubilized, the temperature is allowed to drop to 25° C. and 0.99 g of the graft (Leu)₃NH₂, previously solubilized in 2 ml of DMF, 0.068 g of 4-dimethylaminopyridine, previously solubilized in 1 ml of DMF, and 0.43 g of diisopropylcarbodiimide, previously solubilized in 0.5 ml of DMF, are added in succession. After 8 hours at 25° C., with stirring, the reaction medium is poured into 280 ml of water containing 15% of sodium chloride and hydrochloric acid (pH 2). The precipitated polymer is then recovered by filtration and washed with 0.1 N hydrochloric acid and then with chloroform. The polymer is subsequently dried in an oven under vacuum at 40° C. to give a yield in the order of 80%. The grafting rate estimated by proton NMR is about 8%.

### EXAMPLE 2

### Preparation of Polymers P2 to P5

Polymers P2 to P5 are prepared under the same conditions as those used for polymer P1 except that the grafting rate and the nature of the (oligo)amino acid are varied.

The grafts (Val)₃NH₂ and (Phe)₂NH₂ are marketed in the HCl form by BACHEM. They are used after deprotonation with triethylamine.

The graft (Leu)NH₂ is marketed by ALDRICH.

The characteristics of the synthesized polymers are collated in the Table below.

TABLE 1

| | | Grafting rate | Mn** g/mol |
|---|---|---|---|
| Polymer | Graft* | (NMR) | (equiv. PMMA) |
| P1 | (Leu)₃NH₂ | 8% | 19,500 |
| P2 | (Leu)₃NH₂ | 21% | 17,300 |
| P3 | (Val)₃NH₂ | 22% | 18,300 |
| P4 | (Phe)₂NH₂ | 22% | 17,300 |
| P5 | (Leu)NH₂ | 40% | 29,300 |

*Leu: L-leucine, Val: L-valine, Phe: L-phenylalanine
**Mn: number-average molecular weight

In all cases the polymers are dispersible in water at pH 7.4 in a concentration of about 20 mg/ml and are limpid. Analysis of these polymers by light scattering shows that, depending on the grafting rate and the concentration, they form objects of 20 to 200 nm.

### EXAMPLE 3

### Adsorption of a Dye onto Polymers P1, P3 and P4

According to one of the objects of the invention, the polymers can be used in water and associate or encapsulate an active principle (in the form of a colloidal or non-colloidal suspension). For this application, it is demonstrated in the following experiment that polymers P1, P3 and P4 are capable of associating or encapsulating a standard dye.

The study is carried out in the following manner: The polymers are solubilized in an aqueous solution of pH 7 (phosphate buffer) and 5 mg of the dye called Orange OT (Rn CAS: 2646-17-5) are added. The solutions are left in an ultrasonic bath for one hour to effect the association. The solutions are then centrifuged to remove the non-associated dye and the optical density (OD) is measured at the λmax of the dye (495 nm) after dilution. The experiment with polyglutamate on its own serves as a reference.

TABLE 2

| Polymer | Polymer concentration | Relative induced OD |
|---|---|---|
| P1 | 15.6 mg/ml | 0.27 |
| P3 | 8 mg/ml | 0.27 |
| P4 | 8 mg/ml | 0.21 |
| Polyglutamate | 25 mg/ml | 0.02 |

This experiment shows that these polymers are capable of associating a water-insoluble dye.

### EXAMPLE 4

### Adsorption of Insulin

An aqueous solution of pH 7.4 containing 10 mg of polymer P2 per milliliter and 200 IU of insulin (7.4 mg) is prepared. The solutions are incubated for two hours at room temperature and the free insulin is separated from the associated insulin by ultrafiltration (threshold at 100 kDa, 15 minutes under 10,000 G at 18° C.). The free insulin recovered from the filtrate is then quantitatively determined by HPLC (high performance liquid chromatography) and the amount of associated insulin is deduced. The amount of associated insulin is 110 IU. In comparison, the amount of insulin associated with the reference polyglutamate is zero.

1. Polyamino acid comprising aspartic units and/or glutamic units, some of which carry at least one graft, characterized in that:
at least one of these grafts is bonded to an aspartic or glutamic unit via an amide linkage,
at least some of these grafts comprising one or more (oligo)amino acids, excluding the grafts carrying at least one carboxylic diacid cyclizable to an anhydride,
and the “amino acid” unit(s) in the (oligo)amino acid is (are) selected from those having an alkyl or aryl group in the alpha position, and preferably from those belonging to the group comprising alanine, valine, leucine, isoleucine and phenylalanine.

2. Polyamino acid according to claim 1, characterized in that the oligoamino acid or (oligo)amino acids consists (each consist) of mutually identical “amino acid” units.

3. Polyamino acid according to claim 1 or 2, characterized by general formula (I) below: 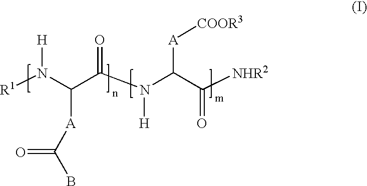
in which:
R¹ is H, a linear C2 to C10 alkyl or branched C3 to C10 alkyl, a benzyl or a terminal “amino acid” unit;
R² is H, a linear C2 to C10 acyl or branched C3 to C10 acyl group or a pyroglutamate;
R³ is H or a cationic entity preferably selected from the group comprising:
metal cations advantageously selected from the subgroup comprising sodium, potassium, calcium and magnesium,
organic cations advantageously selected from the subgroup comprising:
cations based on amine,
cations based on oligoamine,
cations based on polyamine (polyethylenimine being particularly preferred),
and cations based on amino acid(s) advantageously selected from the class comprising cations based on lysine or arginine,
and cationic polyamino acids advantageously selected from the subgroup comprising polylysine and oligolysine;
the n groups B independently of one another are each a monovalent radical of the following formula: 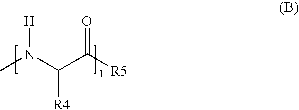
in which:
R⁴ is a methyl (alanine), isopropyl (valine), isobutyl (leucine), sec-butyl (isoleucine) or benzyl (phenylalanine);
and R⁵ is a group selected from OH, NH₂, a C1 to C5 alkoxy group and a benzyloxy;
A independently is —CH₂— (aspartic unit) or —CH₂—CH₂— (glutamic unit);
n/(n+m) is defined as the molar grafting rate and varies from 0.5 to 100 mol %;
n+m varies from 3 to 1000 and preferably between 30 and 300;
and 1 varies from 1 to 6.

4. Polyamino acid according to claim 1, characterized in that all the amino acids constituting the (oligo)amino acid are of the L type.

5. Polyamino acid according to claim 1, characterized in that it consists of an alpha-L-glutamate or alpha-L-glutamic homopolymer.

6. Polyamino acids according to claim 1, characterized in that it consists of an alpha-L-aspartate or alpha-L-aspartic homopolymer.

7. Polyamino acid according to claim 1, characterized in that it consists of an alpha-L-aspartate/alpha-L-glutamate or alpha-L-aspartic/alpha-L-glutamic copolymer.

8. Polyamino acid according to claim 1, characterized in that the distribution of the aspartic and/or glutamic units carrying grafts is such that the resulting polymers are either random or of the block type or of the multiblock type.

9. Polyamino acid according to claim 1, characterized in that its molecular weight is between 2000 and 100,000 g/mol and preferably between 5000 and 40,000 g/mol.

10. Polyamino acid according to claim 1, characterized in that the molar grafting rate is between 2 and 70% and preferably between 5 and 40%.

11. Pharmaceutical, cosmetic, dietetic or phytosanitary composition comprising at least one polyamino acid according to claim 1.

12. Composition according to claim 11, characterized in that it comprises at least one active principle.

13. Pharmaceutical, cosmetic, dietetic or phytosanitary composition according to claim 11 in particular, comprising at least one polyamino acid containing aspartic units and/or glutamic units, some of which carry at least one graft:
at least one of these grafts being bonded to an aspartic or glutamic unit via an amide linkage,
at least some of these grafts comprising one or more (oligo)amino acids,
and the grafts carrying at least one carboxylic diacid cyclizable to an anhydride being excluded,
characterized in that it comprises at least one active principle associated with the polyamino acid(s) by one or more bonds other than one or more covalent chemical bonds.

14. Composition according to claim 12 or 13, characterized in that the active principle is a protein, a glycoprotein, a protein bonded to one or more polyalkylene glycol chains {preferably polyethylene glycol (PEG) chains: “PEGylated protein”}, a polysaccharide, a liposaccharide, an oligonucleotide, a polynucleotide or a peptide.

15. Composition according to claim 12 or 13, characterized in that the active principle is a small hydrophobic, hydrophilic or amphiphilic organic molecule.

16. Composition according to claim 11, 12 or 13, characterized in that it can be administered by the oral, parenteral, nasal, vaginal, ocular, subcutaneous, intravenous, intramuscular, intradermal, intraperitoneal, intracerebral or buccal route.

17. Composition according to claim 11, 12 or 13, characterized in-that it is in the form of a gel, an emulsion, micelles, nanoparticles, microparticles, a powder or a film.

18. Composition according to claim 11, 12 or 13, characterized in that it is a colloidal suspension of nanoparticles and/or microparticles and/or micelles of polyamino acids in an aqueous phase.

19. Composition according to claim 11, 12 or 13, characterized in that it is in the form of a solution in a biocompatible solvent and in that it can be injected by the subcutaneous or intramuscular route or into a tumor.

20. Composition according to claim 11, 12 or 13, characterized in that it is capable of forming a deposit at the injection site.

21. Process for the preparation of:
drugs, particularly for administration by the oral, nasal, vaginal, ocular, subcutaneous, intravenous, intramuscular, intradermal, intraperitoneal or intracerebral route, it being possible in particular for the active principles of these drugs to be proteins, glycoproteins, proteins bonded to one or more polyalkylene glycol chains {e.g. polyethylene glycol (PEG) chains, in which case the term “PEGylated” proteins is used}, peptides, polysaccharides, liposaccharides, oligonucleotides, polynucleotides and small hydrophobic, hydrophilic or amphiphilic organic molecules;
and/or nutriments;
and/or cosmetic or phytosanitary products,
characterized in that it consists essentially in using at least one polyamino acid according to claim 1 and/or the composition according to claim 11, 12 or 13.
